(19) United States
(12) Patent Application Publication
(10) Pub. No.: US 2015/0057650 A1
(43) Pub. Date: Feb. 26, 2015

(54) DEVICE AND METHOD FOR INDUCING BLOOD COAGULATION AND REDUCING INFECTION WITH STERILIZED HEATED AIR AND LOCALLY DIRECTED LIGHT OR OTHER ELECTROMAGNETIC RADIATION

(71) Applicant: Morton Grosser, Menlo Park, CA (US)

(72) Inventor: Morton Grosser, Menlo Park, CA (US)

(21) Appl. No.: 14/465,102

(22) Filed: Aug. 21, 2014

Related U.S. Application Data
(60) Provisional application No. 61/868,450, filed on Aug. 21, 2013.

(51) Int. Cl. A61B 18/04; A61L 9/20; A61L 9/18

(52) U.S. Cl. CPC A61B 18/04; A61L 9/18; A61L 9/20; A61B 2018/00589
USPC 606/27

(57) ABSTRACT

The device for inducing rapid blood coagulation comprises an enclosure, a fan within the enclosure, a heater within the enclosure, and a light source within the enclosure. The fan draws air into the enclosure past the heater and past the light source. The light source sterilizes the airstream while the heater heats the airstream to a temperature suitable for inducing blood coagulation.

### CROSS-REFERENCE TO RELATED APPLICATION

This application claims the benefit of provisional application No. 61/868,450 (Attorney Docket No. 45167-703.101), filed on Aug. 21, 2013, the full disclosure of which is incorporated herein by reference.

### BACKGROUND OF THE INVENTION

1. Field of the Invention

The present invention relates generally to medical devices and methods. More particularly, the present invention relates to a hand-held device for producing a heated, sterilized airstream optionally combined with UV or other electromagnetic radiation (EMR) for inducing blood coagulation and/or sterilization.

Hemostasis refers to the cessation of bleeding. Hemostasis occurs naturally as a result of the clotting cascade where a series of clotting factors in the blood are activated sequentially when the blood is exposed to oxygen in air. While natural clotting is sufficient to stop most minor bleeding, traumatic injuries and surgery usually require some type of intervention to enhance or supplement natural clotting.

Of particular interest to the present invention, it has been found that heating blood will accelerate the natural clotting process. While the heat is often provided using a laser or other light source, it has also been recognized that heated air and air plasmas when directed at a wound can effectively accelerate coagulation.

The use of hot air for promoting blood coagulation in patients, however, has not become widespread for a number of reasons such as a lack of specific tools, particularly low cost tools which could be used for minor cuts in the home; concern over sterility of the heated airstream being delivered; convenience; cost, and other factors.

Therefore, it would be desirable to provide low cost, easy to use, portable tools capable of generating a heated airstream optionally combined with UV light or other EMR for enhancing blood coagulation with a minimum risk of bacterial or viral contamination. At least some of these objectives will be met by the inventions described hereinbelow.

2. Description of the Background Art

Kuo et al. (2009) New J. Phys. 115016 describes a low temperature air plasma torch that can be used to induce rapid blood coagulation. A handheld cautery device that generates a non-sterile heated airstream is described in U.S. Publ. 2011/0196360. Medical devices for delivering hot air to patients for a variety of purposes are described in U.S. Pat. Nos. 5,620, 440 and 7,585,295. Other portable hot air generators are described in U.S. Pat. Nos. 2,859,327; 3,492,462; 3,668,370; 4,634,839; 6,370,326; 6,683,285; and 6,928,235. A hand-held device for delivering light from LEDs to a patient's skin is described in U.S. Pat. No. 8,435,273.

### SUMMARY OF THE INVENTION

In a first aspect of the present invention, a device for inducing rapid blood coagulation comprises a hand-held enclosure having an interior, an air inlet, and an air outlet. A fan is disposed within the interior and is configured to draw an airstream in through the air inlet and to deliver said airstream out through the air outlet. A heater is disposed within the interior of the hand-held enclosure and is configured to heat the airstream as said airstream passes through the interior. In addition to the heater, ultraviolet (UV) light and/or other electromagnetic radiation sources are disposed within the interior and are configured to sterilize the airstream and/or the tissue site as the airstream passes through the interior. The heated, sterilized airstream is suitable for inducing blood coagulation in cases of cuts and wounds in a patient's skin as well as internal bleeding which may result from accidents and/or surgical treatments.

The hand-held device will typically further comprise a battery or power cell within the enclosure which is connected to power the fan, the heater, and the light source. In this way, the device can be completely self-contained and ready for immediate use. The battery or power cell may be replaceable but will more usually be rechargeable so that the device may be optionally held in a charging unit so that it is ready for use at all times. In other instances, however, the device may further include a line cord and internal power supply which allows the device to be run from line current. In still other instances, of course, the device may be configured to operate alternatively from an internal battery or power cell or from external line current.

In specific examples of the device, the fan may comprise an axial flow fan, such as a series of fan or compressor blades, which produce an axially flowing airstream within the hand-held enclosure. In other instances, the fan may comprise a cross-flow fan which has air intakes on the side of the enclosure to draw the airstream radially inwardly and produce an axial outlet airstream.

The heater may comprise any one of a variety of convective heaters which produce high temperature surfaces over which the airstream flows. Exemplary heaters include electrical resistance heaters, such as nichrome coil heaters, ceramic plate heaters, and the like. The heated surfaces will typically be maintained at a temperature in the range from about 200° C. to 400° C., and the airstream flow will be selected to produce an outlet airstream having a temperature in the range from about 40° C. to 58° C. The flow rate of the airstream will typically be in the range from 0.05 m³/sec to 0.5 m³/sec, where the ratio of volumetric air flow to heated surface area being further selected to maintain a temperature in the desired range set forth above.

In specific embodiments, the air flow fan will be controllable to deliver an airstream flow rate in the range from about 0.01 m³/sec to 0.8 m³/sec to, typically from 0.05 m³/sec to 0.5 m³/sec. The heater will be controllable to deliver an airstream having a temperature from 40° C. to 58° C., where the exact amount of energy delivered by the heater is selected based on all relevant parameters including airstream flow rate, heater surface area, and the like.

The light source is preferably configured to deliver ultraviolet or other wavelengths capable of disinfecting the airstream. Most preferably, ultraviolet light having a short wavelength, typically from 253 nm to 290 nm, referred to as UVC, will be used. In the specific embodiments, the UV light will be produced by light emitting diodes (LEDs) emitting at 280 nm which are situated at some point along the airstream flow path. Preferably, the LED or other light source will be located at or close to the air inlet of the enclosure so that the air is sterilized before or as it enters the hand-held enclosure. In this way, the risk of contaminating the interior of the hand-held enclosure is reduced.

In other embodiments, the device can use a narrow spectrum of UV light selected to destroy drug-resistant bacteria but which is safe for human exposure. In particular, narrow-spectrum UV light of 207 nm effectively reduces wound infections without damaging tissue. This device can incorporate a source or sources of UV light of 207 nm frequency, emitted in parallel with the stream of heated air, to directly sterilize a wound or area of trauma while augmenting coagulation together with the heated air. The same or an alternative light source can be used to sterilize the heated air.

Blue light having a wavelength in the range from 405 nm to 470 nm can also be used to sterilize the wound and/or sterilize the heated airstream. This light wavelength can effectively sterilize both gram-positive and gram-negative bacteria, as well as resistant colonies of Staphylococcus aureus. The device of the present invention can incorporate a source or sources of 405 to 470 nm frequency light, emitted in parallel with the stream of heated air, to kill bacteria in or near a wound or area of trauma while augmenting coagulation.

In a second aspect of the present invention, a method for inducing rapid coagulation or hemostasis in a patient comprises providing a hand-held enclosure having an interior. An ambient airstream is flowed through the interior from an inlet end to an outlet end at a flow rate in the range from 0.05 m³/sec to 0.5 m³/sec. The ambient air is heated to a temperature at the outlet in the range from 40° C. to 58° C. Light is directed at the air flowing through the enclosure to sterilize the ambient air before it flows from the outlet. The volume and temperature of the air are selected to be sufficient to induce coagulation of blood in a body wound. As sterilized air is being used, the risk of infection is greatly reduced.

In exemplary embodiments of the methods of the present invention, the UV light is delivered at a dose sufficient to kill at least 90% of pathogens present in the ambient air, preferably at least 95% of the pathogens, and more preferably at least 99% of the pathogens. In further specific embodiments, the air flow will be recirculated past the directed light to enhance sterility. In still further specific embodiments, the use of light for sterilization can be combined with filtering the air flow to still further reduce pathogens present in the airstream.

A particularly preferred device for inducing rapid hemostasis and coagulation comprises a hand-held enclosure having an interior, an air inlet end at a proximal end of the enclosure, and an air outlet at a distal end of the enclosure. A plurality of ultraviolet (UV) light emitting diodes are disposed about the air inlet. A cylindrical battery (or battery in a cylindrical holder) is disposed in a proximate portion of the interior of the enclosure where the battery defines an annular air flow path between an exterior surface of the battery or holder and an interior wall of the enclosure. An in-line axial fan is disposed in the interior on a distal side of the battery, and an in-line flow-through heater is disposed in the interior of the enclosure on a distal side of the axial fan and a proximal side of the air outlet. Typically, this device will further comprise a temperature control component and an air rate flow component on an exterior of the hand-held enclosure.

### BRIEF DESCRIPTION OF THE DRAWINGS

FIG. 1 is an isometric view of a rapid coagulation device constructed in accordance with the principles of the present invention.

FIG. 2 is a cross-sectional view of the rapid coagulation device of FIG. 1.

FIG. 3 is a block diagram showing the air flow pattern of the rapid coagulation device of the present invention.

### DETAILED DESCRIPTION OF THE INVENTION

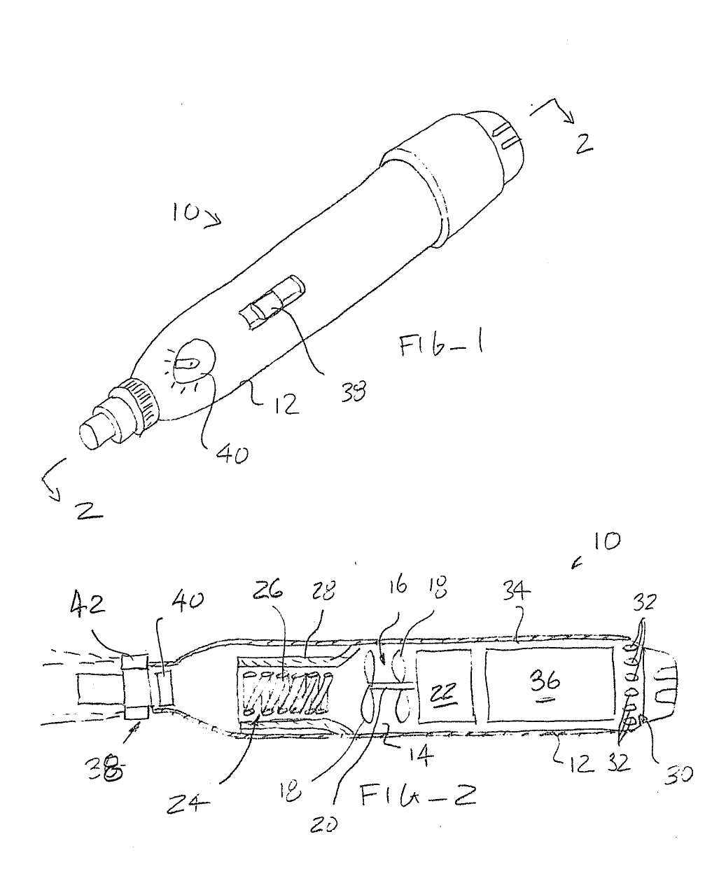

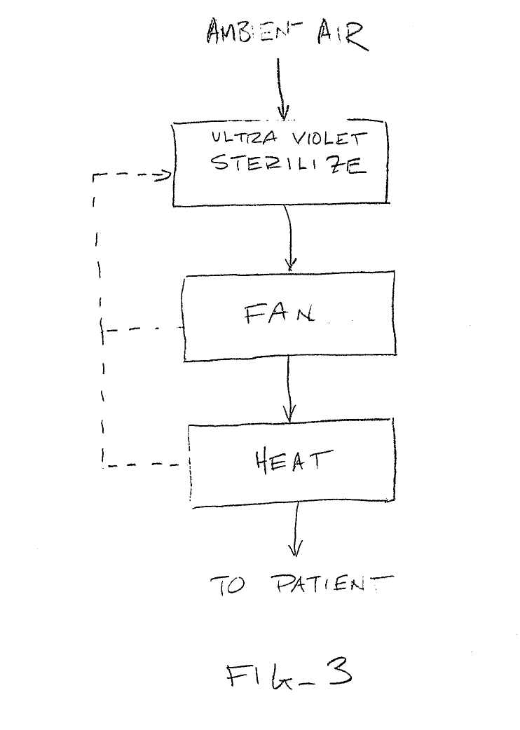

Referring to FIG. 1, a rapid coagulation device 10 constructed in accordance with the principles of the present invention comprises a hand-held enclosure 12 having an interior space 14. A fan assembly 16 typically comprises a plurality of fan blades 18 mounted on a rotatable shaft 20 which is driven by a motor 22.

A heating unit 24 usually comprises a plurality of coils 26, typically nichrome coils or other electrically resistive coils which can be heated within the temperature ranges described above. The heating coils 26 are typically placed within a flow tube 28 which funnels and directs flow from the fan 16 past the heating coils in the desired flow rate range, also as set forth above. Air enters the interior 14 of the enclosure through an air inlet 30 at a proximal end of the device 10. A plurality of sterilizing light sources, such as LED's 32, are mounted in or about the air inlet so that ambient air is sterilized upon entry into the interior 14 of the housing 12. The air preferably flows past an annular flow space 34 surrounding a battery 36 as it is drawn inwardly by the fan 16. Air leaving the fan 16 flows past the heating coils 26 and out through an outlet 34 which is typically adjustable to control the air flow within a desired range. Alternatively or additionally, the air flow may be adjusted using a speed control 38 on the exterior of the housing. Additionally, temperature may be varied using a temperature control knob 40, also on the exterior of the housing 12, which will typically adjust the power being delivered to the heating coils 26. Optionally, a temperature sensor (not shown) may be positioned to measure the temperature of the heated air exiting the air outlet, and feedback or other control means may be provided for maintaining a target temperature with the target range defined above.

Referring now to FIG. 3, preferred and exemplary air flow patterns will be described. Ambient air enters the interior 14 of enclosure 12 where it will immediately pass by the ultraviolet sterilizing lights 32. Flow is induced by the fan 16 past the heating coils 26 and out through the outlet 38 to the patient. Optionally, a portion of the airstream may be re-circulated from the fan and/or from the heater to flow back past the ultraviolet sterilization lights in order to increase the removal of pathogens, as shown in broken line on FIG. 3. Further optionally, a filter 40 may be provided at the outlet 38 to further remove bacteria and other contaminants. Further additionally, a UV, blue, or other light source may be provided for directly irradiating the wound treatment site to provide direct sterilization. This light source will preferably operate in the specific wavelengths set forth above.

While the exemplary embodiments have been described in some detail for clarity of understanding and by way of example, a variety of additional modifications, adaptations and changes may be clear to those of skill in the art. Hence, the scope of the present invention is limited solely by the appended claims.

What is claimed is:

1. A device for rapid blood coagulation, said device comprising:
a handheld enclosure having an interior, an air inlet, and an air outlet;
a fan within the interior and configured to draw an airstream in through the air inlet and to deliver said airstream out through the air outlet;
a heater within the interior and configured to heat the airstream as said airstream passes through the interior; and
a light source within the interior and configured to sterilize the airstream as the airstream passes through the interior.

2. A device as in claim 1, further comprising a battery or cell within the enclosure connected to power the fan, heater, and light source.

3. A device as in claim 1, further comprising a line cord adopted to connect the device to the current.

4. A device as in claim 1, wherein the fan comprises an axial flow fan.

5. A device as in claim 1, wherein the fan comprises a cross-flow fan.

6. A device as in claim 1, wherein the heater comprises electrically resistive coils.

7. A device as in claim 1, wherein the heater comprises a ceramic heater.

8. A device as in claim 1, wherein the light source is configured to deliver ultraviolet light.

9. A device as to claim 8, wherein the light source comprises light emitting diodes (LEDs).

10. A device as in claim 1, wherein the light source is selected to deliver UV light having a wavelength of 207 nm.

11. A device as in claim 1, wherein the light source is selected to deliver blue light having a wavelength in the range from 405 to 470 nm.

12. A device as in claim 1, wherein the fan is controllable to deliver an airstream having a flow rate of 0.05 m³/sec to 0.5 m³/sec.

13. A device as in claim 1, wherein the heater is controllable to deliver an airstream having a temperature from 40° C. to 58° C.

14. A method for inducing rapid hemostasis, said method comprising:
providing a hand-held enclosure having an interior:
flowing ambient air through the interior from an inlet to an outlet at a flow rate in the range from 0.05 m³/sec to 0.5 m³/sec;
heating the ambient air to a temperature at the outlet in the range from 40° C. to 58° C.;
directing light at the air flowing through the enclosure to sterilize the ambient air before it flows from the outlet and/or directly at a wound site to sterilize said site;
wherein the volume and temperature of the air are sufficient to induce coagulation of blood in a body wound.

15. A method as in claim 14, wherein the light is UV light delivered at a dose sufficient to kill at least 90% of the pathogens present in the ambient air.

16. A method as in claim 15, wherein the dose is sufficient to kill at least 95% of the pathogens.

17. A method as in claim 16, wherein the dose is sufficient to kill at least 99% of the pathogens.

18. A method as claim 14, wherein the air flow is recirculated past the directed light to enhance sterility.

19. A method as in claim 14, wherein the delivered light comprises UV light having a wavelength of 207 nm.

20. A method as in claim 14, wherein the delivered light comprises light having a wavelength in the range from 405 to 470 nm A method as to claim 13, further comprising filtering the air flow.

21. A device for rapid hemostasis coagulation, said device comprising:
a hand-held enclosure having an interior, an air inlet end at a proximal end and an air outlet at a distal end;
a plurality of UV light emitting diodes disposed about the air inlet;
a cylindrical batter disposed in a proximal portion of the interior of the enclosure, said battery delivering an annular air flow path between an exterior surface of the battery and an inner wall of the enclosure;
an in-line axial fan disposed in the interior on a distal side of the battery;
an in-line flow-through heater disposed in the interior of the enclosure on a distal side of the axial fan and a proximal side of the air outlet.

22. A device as in claim 19, further comprising a temperature control and an air flow role control on the exterior of the hand-held enclosure.
